(19) United States
(12) Patent Application Publication
(10) Pub. No.: US 2013/0309190 A1
(43) Pub. Date: Nov. 21, 2013

(54) HAIR COSMETIC AND STYLING COMPOSITIONS BASED ON MALEIC ACID COPOLYMERS AND POLYAMINES

(71) Applicants: Emmanuel Dimotakis, Oradell, NJ (US); Jim Mitchell Singer, South Orange, NJ (US); Hy Si Bui, Piscataway, NJ (US); Jean-Thierry Simonnet, Mamaroneck, NY (US)

(72) Inventors: Emmanuel Dimotakis, Oradell, NJ (US); Jim Mitchell Singer, South Orange, NJ (US); Hy Si Bui, Piscataway, NJ (US); Jean-Thierry Simonnet, Mamaroneck, NY (US)

(73) Assignee: L'OREAL S.A., Paris (FR)

(21) Appl. No.: 13/471,983

(22) Filed: May 15, 2012

(51) Int. Cl. A61K 8/84; A61Q 5/06

(52) U.S. Cl. 424/70.17

(57) ABSTRACT

Disclosed are cosmetic compositions which include hair cosmetic or styling compositions, comprising: a maleic acid copolymer; a polyamine selected from polyamino acids and aminated polysaccharides; a neutralizer; and water, wherein the polyamine is present in an amount less than 0.1% by weight based on the total weight of the composition, and methods of making and using the compositions to impart or maintain hair style.

### BACKGROUND OF THE INVENTION

Under high humidity conditions, hair tends to absorb moisture causing it to be less manageable, which makes it more difficult to shape and style hair even when using hair styling compositions. Frizzy hair is particularly prone to problems when exposed to higher humidity. Applying a moisture barrier on the hair is known to help to keep moisture out of the hair allowing for more efficient hair shaping and maintenance of hair shape, even in high humidity conditions. These types of haircare products that are the most commonly available on the cosmetics market are gels, mousses, and spray compositions that contain one or more polymer resins, known as fixing materials.

Spray compositions, especially aerosols, typically contain at least one volatile organic compound (VOC). For essentially ecological reasons and governmental regulations in various countries, it is sought or even necessary to reduce the amount of volatile organic compounds (VOCs) present in the composition. To reduce the amount of VOC and to obtain a low-VOC aerosol device, the organic solvents, for instance ethanol and dimethyl ether, are partially replaced with water, and concomitantly, use of more water-soluble fixing resins such as maleic acid copolymers.

By way of illustration of the forementioned haircare products, GB 1,603,321 and 1,603,323 teach treatment of hair using a combination of cationic polymers and anionic polymers derived from carboxylic acids, e.g., acrylic or methacrylic acid, and maleic anhydride. U.S. Pat. No. 5,176,898 is directed to an aqueous hairspray composition having low VOC content, and which contains in addition to a volatile silicone, a water-soluble resin, e.g., polyvinyl methacrylate/maleic anhydride copolymer, and the butyl ester thereof. U.S. Pat. No. 7,041,281 teaches personal care formulations such as hair care formulations that contain a so-called natural feel polymer that contains repeating units of a maleic anhydride alkyl half-ester or full acid, maleamic acid and maleimide. U.S. Pat. No. 7,837,983 teaches hair care compositions for mending split ends that contain a polyelectrolyte complex between a cationic polyquaternium polymer and an anionic copolymer such as a polyvinylmethyl/maleic acid copolymer, or a butyl ester thereof. WO/2011/143060 discloses compositions, including hairsprays that contain modified maleic anhydride copolymers such as including Aquaflex XL-30. WO 2012/021472 teaches hair care compositions that contain a polyacid and a polyamine.

However even at very low to zero VOCs, problems related with moisture sensitivity of fully water-based products have been found to be problematic from the standpoints of moisture sensitivity and eco-toxicity depending on the polymer and regional regulations. These issues aside, the ability to maintain the shape of hair or to re-style the hair without reapplication of product, humidity resistance, hair strengthening and color fading remain as additional areas for improvement, particularly in connection with maleic-based polymers.

### BRIEF SUMMARY OF THE INVENTION

A first aspect of the present invention is directed to a cosmetic composition, comprising: a maleic acid copolymer; a polyamine selected from polyamino acids and aminated polysaccharides; a neutralizer; and water, wherein the polyamine is present in an amount less than 0.1% by weight based on the total weight of the composition. In preferred embodiments, the compositions are formulated as hair cosmetic or styling compositions. In other preferred embodiments, the compositions are free of silicones.

A second aspect of the present invention is directed to a method of imparting or maintaining shape of hair, comprising applying to hair a cosmetic composition, comprising: a maleic acid copolymer; a polyamine selected from polyamino acids and aminated polysaccharides; a neutralizer; and water, wherein the polyamine is present in an amount less than 0.1% by weight, based on the total weight of the composition.

### BRIEF DESCRIPTION OF THE DRAWINGS

FIGS. 1A and B are schematic diagrams that illustrate the type of inter-polymer interactions that may occur between the maleic acid copolymer and the polyamine (e.g., polylysine in FIG. 1A and chitosan in FIG. 1B).

### DETAILED DESCRIPTION

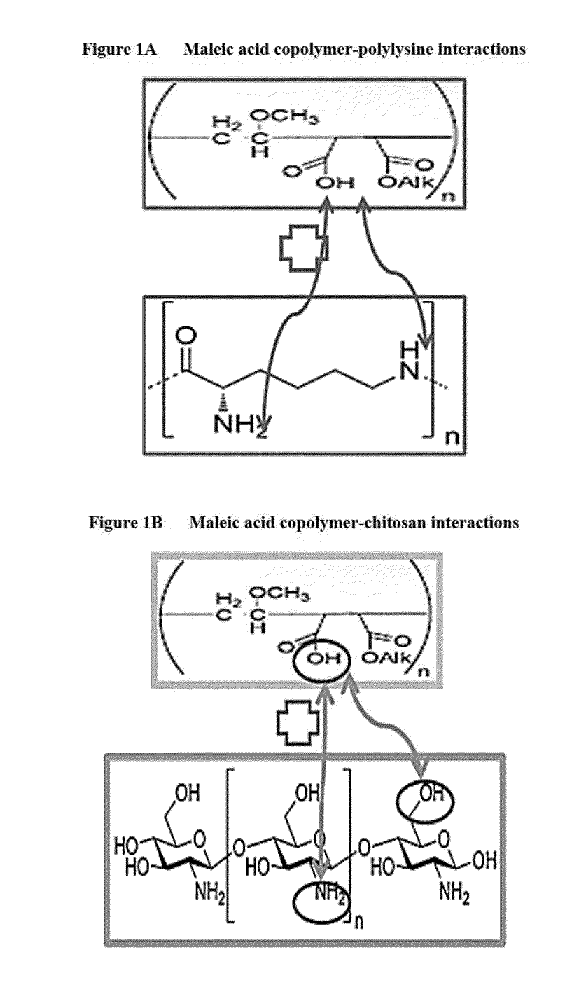

Aside from being relatively non-ecotoxic, the compositions of the present invention can be easily formulated in a variety of galenic forms (e.g., hair sprays, gels, pastes, waxes, creams, mousses) in water-based or low alcohol/water solutions, to facilitate hair styling. To Applicants' unexpected surprise, notwithstanding the relatively small amounts of the polymers and particularly the polyamine that are present, when applied to the hair, the inventive compositions effectively seal the cuticle, and thus provide an advantage in terms of increased resistance to humidity (as demonstrated by curl retention, anti-frizz, etc.). Additional advantages may include increased resistance to color fading, increased strengthening of the hair fibers, and maintenance of hair style without re-application of the composition. Without intending to be bound by any particular theory of operation, and as illustrated in FIGS. 1A and B, Applicants believe that these advantages of the compositions are attributable, at least in part, to inter-polymeric interactions that occur and which serve to provide an effective seal on and protection of the individual hair fibers. Further, the inventive compositions remain stable even during storage, and exhibit no precipitation of the polymers.

The term “comprising” (and its grammatical variations) as used herein is used in the inclusive sense of “having” or “including” and not in the exclusive sense of “consisting only of”. The terms “a” and “the” as used herein are understood to encompass the plural as well as the singular.

Other than in the operating examples, or where otherwise indicated (and not contraindicated), all numbers expressing quantities of ingredients and/or reaction conditions are to be understood as being modified in all instances by the term “about” which encompasses +10%.

At least one” as used herein means one or more and thus includes individual components as well as mixtures/combinations thereof.

The term “lipophilic” means those compounds which are soluble in oils and are either completely or partially insoluble in water. In accordance with the present disclosure, the solubility of the lipophilic compounds is preferably less than 5% by weight, more preferably less than 1% by weight, even more preferably less than 0.5% by weight and better still, less than 0.1% by weight in water at 25° C. and at atmospheric pressure.

The term “stable” as used herein means that the composition does not exhibit phase separation.

The term “sealing” (and its grammatical variations) as used herein means to provide a protective barrier over the exterior of a keratinous substrate such as hair in order to inhibit water from penetrating into and escaping from the keratinous substrate.

“Substituted,” as used herein, means comprising at least one substituent. In addition to other specific substituents disclosed herein, non-limiting examples of substituents include atoms, such as oxygen atoms and nitrogen atoms, as well as functional groups, such as hydroxyl groups, ether groups, alkoxy groups, acyloxyalkyl groups, oxyalkylene groups, polyoxyalkylene groups, carboxylic acid groups, amine groups, acylamino groups, amide groups, halogen-containing groups, ester groups, thiol groups, sulphonate groups, thiosulphate groups, siloxane groups, and polysiloxane groups. The substituent(s) may be further substituted.

### Maleic Acid/Anhydride Copolymers

For purposes of the present invention, the term “maleic acid copolymer” refers to and embraces copolymers of maleic acid or maleic anhydride, or a cosmetically acceptable derivative of maleic acid or maleic anhydride, and at least one additional comonomer copolymerizable therewith. Maleic acid copolymers are known in the cosmetics art, e.g., in hair care, as “fixing” polymers or agents, which as used herein refers to a polymer that makes it possible to give or impart a shape to the hair and/or to hold or maintain the hair in a given shape.

Representative examples of the co-monomers that are polymerizable with the maleic acid, anhydride or derivative thereof, include vinyl esters (e.g., alkyl (e.g., C1-C4) vinyl esters such as methyl vinyl esters), vinyl ethers (e.g., alkyl (e.g., C1-C4) vinyl ethers such as methyl vinyl ether and isobutyl vinyl ether), vinyl halides (e.g., alkyl (e.g., C1-C4) vinyl halides), phenylvinyl derivatives (e.g., styrene), alpha-olefins (e.g., C2-C20 olefins such as ethylene, propylene, butylene and isobutylene), cycloolefins (e.g., norbornene, 5-methylnorbornene, 5-ethylnorbornene, 5,6-dimethylnorbornene, 5,5,6-trimethylnorbornene, 5-ethylidenenorbornene, 5-phenyl-norbornene, 5-benzylnorbornene, and 5-vinylnorbornene), vinyl triazoles (e.g., 4- and 5-vinyl-substituted 1,2,3-triazoles), and (meth)acrylic acid and esters thereof.

Thus, representative examples of maleic acid/anhydride copolymers that may be useful in the practice of the present invention include methyl vinyl ether-maleic acid copolymer (e.g., available commercially from ISP under the tradename GANTREZ® S (e.g., GANTREZ® S-97 BF; INCI:PVM/MA copolymer)), methyl vinyl ether-maleic anhydride copolymer (e.g., available commercially from ISP under the tradename GANTREZ® AN; INCI:polymethylvinylether/maleic anhydride copolymer), isobutyl vinyl ether-maleic anhydride copolymer, ethylene-maleic anhydride copolymer, isobutylene-maleic anhydride copolymer, styrene-maleic anhydride copolymer, vinyltriazole-maleic anhydride copolymer and vinylnorbornene-maleic anhydride copolymer. The copolymers may contain, in addition to the at least one copolymerizable monomer, both maleic acid and maleic anhydride, or a derivative thereof. U.S. Pat. No. 6,562,928, for example, teaches terpolymers of maleic acid, maleic anhydride and alkylvinylethers.

The maleic acid/anhydride monomer or repeating unit may be derivatized. For example, referring to the representative formula I:

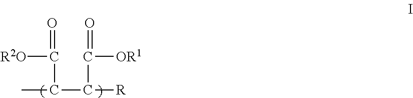

the maleic acid monomer may be partially esterified (i.e., a half-ester wherein either R¹ or R², but not both, represents an alkyl group such as ethyl, isopropyl, butyl) or totally esterified wherein each of R¹ and R² represents an alkyl group (e.g., a C1-C4 alkyl ester group). Copolymers that contain the half-ester are available commercially from ISP under the tradename Gantrez® ES (e.g., Gantrez® ES-225; INCI: ethyl ester of PVM/MA copolymer, and Gantrez® ES-425 and ES-425L; INCI: butyl ester of PVM/MA copolymer).

In yet further embodiments, the carboxyl groups in the maleic acid monomer or repeating unit are converted to a metal salt such as an alkaline metal (e.g., Na) or an alkaline earth metal (e.g., Ca). That is, R¹ and R² each independently represents an alkali metal cation, or together represent an alkaline earth metal cation. Copolymers such as these are available commercially e.g., from ISP under the tradename GANTREZ® MS (INCI: calcium/sodium PVM/MA copolymer).

In other embodiments, the copolymer contains a nitrogen-derivatized maleic acid and/or maleic anhydride repeating unit as represented by formula II or formula III:

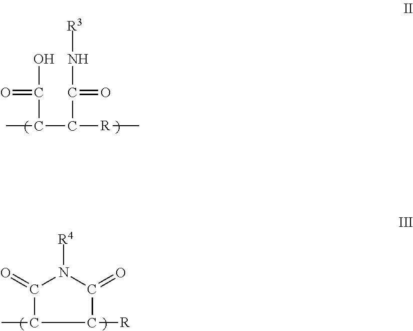

i.e., substituted malemic acid and substituted maleimide respectively, wherein R³ and R⁴ are each a derivatizing group selected from hydrophobic amines (e.g., monofunctional α-unsubstituted primary or secondary monoamines, unsubstituted or substituted with alkyl, aryl, heterocyclic, aromatic, fluoro, silyl, amino, carboxy and halogen, e.g., C1-C40 alkyl NH₂, butylamine, isobutyl amine, and octadecylamine); hydrophilic amines (e.g., hydroxy α-unsubstituted amines e.g., ethanolamine, isopropylamine, n-propanolamine, 3-amino-1-propanol, methoxyethyl amine, and diglycol amine, and alkyl diamines, e.g., 3-(dimethylamino)propylamine, dimethylethylene diamine, N-aminopropyl pyrrolidone, N-aminoethyl pyrrolidone, and 1-(3-aminopropyl)imidazole); and polyether amines (e.g., polyoxyalkylene amine, having the formula (IV):

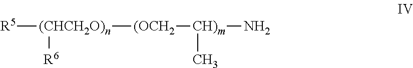

wherein R⁵ and R⁶ are selected from H and alkyl; e.g., R⁵ is CH₃ and R⁶ is H; and R⁵ is CH₃ and R⁶ is CH₃; and n and m are integers from 1-50; e.g., n=32 and m=10); and suitable mixtures thereof. Copolymers such as these may be available commercially from ISP under the tradename AQUAFLEX® (e.g., AQUAFLEX® XL-30 (isobutylene/dimethylaminopropyl maleimide/ethoxylated maleimide/maleic acid copolymer; INCI:polyimide-1; GANTREZ® AQUAFLEX®FX-64; INCI: isobutylene/ethylmaleimide/hydroxyethylmaleimide copolymer. Methods of making these derivatized maleic acid/anhydride copolymers are known in the art and described, for example, in U.S. Pat. Nos. 5,886,194 and 7,041,281.

In yet other embodiments, the maleic acid/anhydride repeating unit or copolymer is derivatized with a so-called modifying reactant moiety which may be represented the formulae: Q—R⁷—NH₂; Z—NH₂; and E—OH, wherein Q is a siloxane, R⁷ is selected from the group consisting of alkyl, cycloalkyl, and aryl groups, Z is selected from the group consisting of hydrogen, alkyl, haloalkyl, aryl, silyl, and tertiary amine groups having the structure NR⁸R⁹, R¹⁰, quarternary amine groups having the structure +NR⁸R⁹R¹⁰R¹¹A⁻, and polyether groups having the structure (V)

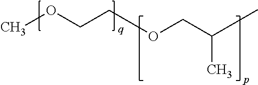

wherein R⁸, R⁹, R¹⁰, and R¹¹ are independently selected from the group consisting of alkyl, cycloalkyl, and aryl groups, A⁻ is an anion, q and p are integers independently ranging from about 1 to about 10,000, E is a siloxane or is selected from the group consisting of hydrogen, alkyl, haloalkyl, aryl, silyl, and amino groups. Maleic acid copolymers produced using these modifying reactant moieties are described in WO 2011/143060. Such copolymers might provide various advantages to styling performance in terms of balancing properties.

In general, the maleic acid/anhydride copolymers possess an acid number/polarity that is less than about 500 (and in some embodiments less than about 400), a refractive index greater than about 1.475, a modulus E′ of from about 0.1 to about 10 GPa (T<Tg), and from about 10 to about 100 MPa (T>Tg), a Tg less than about 120° C., e.g., from about 40 to about 80° C., a molecular weight that may be as high as 3,500,000 (or higher) and an Mc (critical Mw at which Tg is nearly constant with increasing Mw) greater than about 5, and a friction coefficient less than about 1.

In some embodiments, the amount of the maleic acid copolymer generally ranges from about greater than 0 (e.g., about 0.01%) to about 12%, and in some embodiments from greater than 0 to about 8%, and in some embodiments from greater than 0 to about 6%, and in some other embodiments from about 0% to about 4%, on a dry weight basis, based on the total weight of the composition.

### The Polyamine

Polyamines useful in the practice of the present invention include polyamino acids and aminated polysaccharides.

Representative examples of polyamino acids include polyamino acids such as polyaspartic acid, polylysine (p, poly-D-lysine), poly-ornithine, polyhomoarginine, polyarginine, poly-glutamic acid and polyhistidine. In some embodiments, the polyamino acid may be a homo- or a copolymer of cationic amino acid monomers, e.g., poly(lysine/ornithine), or a copolymer composed of amino acid monomers and a different monomer. In some embodiments, the polylysine used is E-Poly-L-lysine (available commercially from JNC America under the tradename PL-25 (E-POLYLYSINE 25% SOLUTION), which is used as a natural preservative in food products, and is safe and non-ecotoxic and may reduce/eliminate preservation in water-based hair styling products. Furthermore, it may reduce/eliminate some cationic conditioning polymers based on use specifications and specific needs. The molecular weight of the polyamino acid generally ranges from about 4700 to about 300,000, or greater.

The polyamine may also be an aminated polysaccharide such as chitosan (known in the art as a linear polysaccharide composed of randomly distributed β-(1-4)-linked D-glucosamine (deacetylated unit) and N-acetyl-D-glucosamine (acetylated unit), or a cosmetically acceptable derivative thereof (e.g., carboxylate, succinamide, etc). Chitosan may be obtained from various sources such as shells and vegetables. Among these compounds, commercially available sources of chitosan include chitosan having a degree of deacetylation of 90.5% by weight, available from Aber Technologies under the tradename KYTAN BRUT STANDARD®, KYATMER® PC (chitosan pyrrolidone-carboxylate, available from Amerchol), and Velsan® Soft (chemical name: chitosan succinamide) and Zenvivv™ and Zenvivo™ Aqua, all available from Clariant/Kitozyme. Yet other examples of chitosan derivatives are described in Paragraphs 228-31 in U.S. Patent Application Publication 2010/236569 A1.

The amount of the polyamine generally ranges from about greater than 0 (e.g., about 0.01%) to less than 0.1%, and in some embodiments from greater than 0 to about 0.099% and in some embodiments from greater than 0 to about 0.09%, and in some embodiments from greater than 0 to about 0.08%, and in some embodiments from greater than 0 to about 0.05%, and in some embodiments from greater than 0 to about 0.04%, and in some other embodiments from about 0.02% to about 0.04%, on a dry weight basis, based on the total weight of the composition.

The ratio of the maleic acid copolymer to the polyamine generally ranges from about from 1/10 to 10,000/1, and in some embodiments from about 1/1 to 500/1 g/g maleic acid copolymer. In yet other embodiments, the ratio is about 0.003-0.20 g/g maleic polymer). Aside from their effectiveness in imparting or maintaining shape of the hair, the relatively low amounts of the polymers allow for flexibility in formulation and cost optimization.

The inventive compositions also contain a neutralizer, which affects pH of the composition so as to allow the maleic acid/anhydride copolymer to remain solubilized. Representative examples of neutralizers useful for this purpose include AMP (aminomethyl propanol), AMPD (aminomethyl propanediol), TIPA (triisopropanol amine), Sodium/Potassium hydroxides, Dimethylsterarylamine, Dimethyl/tallowamine lysine, ornithine, arginine, glutamic and aspartic acid. The degree of maleic polymer neutralization generally ranges from about 5% to about 105% and in some embodiments from about 10% to 100%. The amount of neutralizer is selected on criteria that include the desired pH of the composition, which generally ranges from about 3 to about 9, and in some embodiments, from about 4.5 to about 8. Thus, the amount of neutralizer generally ranges from greater than 0 (e.g., about 0.01%) to about 3%, and in some embodiments from greater than 0% to about 2%, by weight, based on the total weight of the composition.

The compositions of the present invention also contain water, the amount of which varies depending for example, on the type of composition being formulated. Thus, the amount of water generally ranges from about 0.01 to about 99%, and in some embodiments from about 0.01% to about 45%, and in some other embodiments from about 0.01% to about 40%, by weight, based on the total weight of the composition.

Broadly, cosmetic compositions of the present invention fall into two general categories, namely aqueous-based compositions wherein water is present as a sole solvent (e.g., an aqueous solution or aqueous gel) or in combination with at least one water-miscible volatile organic solvent such as ethanol (e.g., an aqueous/alcoholic solution), and multi-phasic compositions such as emulsions (e.g., oil-in-water (O/W), water-in-oil (W/O) or multiple (triple: W/O/W or O/W/O) emulsions). Emulsions contain two or more phases which in addition to the aqueous phase include an oil-phase (that typically contains a liquid fatty phase and/or a fatty substance that is at least partially solid at room temperature (20° C.-25° C.)), wherein the discrete (e.g., continuous and discontinuous) phases are dispersible by the presence of an emulsifier or other cosmetic ingredient with emulsifying properties.

Representative types of hair care compositions, including hair cosmetic and styling compositions, of the present invention include pre-treatments and post-treatments for color protection, conditioning or protection from heat damage, leave-in hair treatments, rinse-off hair treatments, styling products (e.g., gels, creams, milks, pastes, waxes, ointments, serums, foams, hair lotions, mousses, pump-sprays, and aerosol sprays), combination shampoo/styling compositions and hair volumizing compositions.

Accordingly, the cosmetic compositions of the present invention may contain at least one further cosmetically acceptable ingredient, which as those skilled in the cosmetics art will appreciate, is chosen based on several criteria, including for example, the type of product and its intended use and effect, compatibility with the other ingredients, and aesthetic appeal. Representative types of such additional ingredients include volatile organic solvents/compounds, propellants, rheology modifiers (also known as gelling agents or thickeners), emulsifiers and surfactants, fatty phase ingredients such as oils and waxes, photoprotective agents (e.g., U.V. filters), and cosmetically active ingredients. Examples of these ingredients are described herein.

Yet other examples of cosmetically acceptable ingredients that may be present in the inventive compositions include fragrances, preservatives, colorants, glitter, conditioning agents (e.g., cationic polymers, cationic polysaccharides and cationic gums), fillers/powders, emollients, moisturizers, buffers, chelators (such as EDTA and salts thereof, particularly sodium and potassium salts), antioxidants (e.g., BHT, tocopherol), essential oils, proteins, protein hydrolysates, reducing agents, plasticizers, softeners, antifoaming agents, abrasive agents (e.g., pumice, apricot kernel powder), inorganic colloids, peptizing agents, pearlescent agents, penetrants, opacifying agents, silicones and cosmetically active agents, and any other additive or adjuvant conventionally used in cosmetic compositions intended to be applied to the hair. The compositions may further contain another fixing polymer other than the polymers of the invention, provided that it is compatible with the other ingredients therein.

### Volatile Organic Compound/Solvent

Representative examples of organic solvents include C2 to C4 mono-alcohols, such as ethanol, polyols such as C2-C6 glycols e.g., propylene glycol, glycerol, and polyol ethers, acetone, propylene carbonate and benzyl alcohol. The amount of the volatile organic solvent generally ranges from greater than 0 (e.g., about 0.01%) to about 99%, and in some embodiments from greater than 0 to about 55%, and in some embodiments from greater than 0 to about 2%, by weight, based on the total weight of the composition. It is preferred that the amount of volatile organic solvent does not exceed 55%.

### Propellants

Representative examples of propellants include n-butane, isobutane, propane, dimethyl ether (available commercially from Harp Int'l under the tradename HARP DME), C2-C5 halogenated hydrocarbons, e.g., 1,1-difluoroethane (available commercially from DuPont under the tradename DYMEL 152a), difluoroethane, chlorodifluoroethane, chlorodifluoromethane, and mixtures thereof. The amount of the propellant generally ranges from about 1 to about 55%, and in some embodiments from about 1 to about 35%, by weight, and in some embodiments from about 1 to about 20%, by weight and in some embodiments from about 2 to about 15%, by weight based on the total weight of the composition.

### Rheology Modifiers

Broadly, the rheology modifier(s) that may be useful in the practice of the present invention include those conventionally used in cosmetics such as polymers of natural origin and synthetic polymers.

Representative rheology-modifying agents that may be used in the practice of the present invention include nonionic, anionic, cationic, and amphoteric polymers, and other rheology modifiers such as cellulose-based thickeners (e.g., hydroxyethylcellulose, hydroxypropylcellulose, carboxymethylcellulose, cationic cellulose ether derivatives, quaternized cellulose derivatives, etc.), guar gum and its derivatives (e.g., hydroxypropyl guar, cationic guar derivatives, etc.), gums such as gums of microbial origin (e.g., xanthan gum, scleroglucan gum, etc.), and gums derived from plant exudates (e.g., gum arabic, ghatti gum, karaya gum, gum tragacanth, carrageenan gum, agar gum and carob gum), pectins, alginates, and starches, crosslinked homopolymers of acrylic acid or of acrylamidopropane-sulfonic acid, associative polymers, non-associative thickening polymers, and water-soluble thickening polymers.

In some embodiments, the compositions the rheology-modifying agent include a polymer chosen from nonionic, anionic, cationic and amphoteric amphiphilic polymers.

The amphiphilic polymers may, optionally, contain a hydrophobic chain that is a saturated or unsaturated, aromatic or non-aromatic, linear or branched C6-C30 hydrocarbon-based chain, optionally comprising one or more oxyalkylene (oxyethylene and/or oxypropylene) units.

Representative examples of cationic amphiphilic polymers containing a hydrophobic chain include cationic polyurethanes or cationic copolymers comprising vinyllactam units, such as vinylpyrrolidone units.

Representative examples of nonionic amphiphilic polymers containing a hydrophobic chain include the following:

(1) celluloses modified with groups containing at least one saturated or unsaturated, linear or branched C6-C30 hydrocarbon-based chain, for instance hydroxyethylcelluloses modified with groups containing at least one hydrophobic chain as defined previously, such as especially Natrosol® Plus Grade 330 CS(C16 alkyls—sold by the company Aqualon); Bermocoll® EHM 100 (sold by the company Berol Nobel), Amercell Polymer HM-1500 (hydroxyethylcellulose modified with a polyethylene glycol (15) nonylphenyl ether group-sold by the company Amerchol);

(2) hydroxypropyl guars modified with groups comprising at least one hydrophobic chain as defined, for example Jaguar® XC-95/3 (C14 alkyl chain-sold by the company Rhodia Chimie); Esaflor HM 22 (C22 alkyl chain-sold by the company Lamberti); RE210-18 (C14 alkyl chain) and RE205-1 (C20 alkyl chain-sold by the company Rhodia Chimie);

(3) copolymers of vinylpyrrolidone and of hydrophobic monomers containing a hydrophobic chain as defined above, for instance Antaron® or Ganex® V216 (vinylpyrrolidone/hexadecene copolymers); Antaron or Ganex V220 (vinylpyrrolidone/eicosene copolymers), sold by the company I.S.P.;

(4) copolymers of C1-C6 alkyl(meth)acrylates and of amphiphilic monomers containing a hydrophobic chain;

(5) copolymers of hydrophilic (meth)acrylates and of hydrophobic monomers containing at least one hydrophobic chain, for instance the polyethylene glycol methacrylate/lauryl methacrylate copolymer;

(6) polymers with an aminoplast ether skeleton containing at least one fatty chain, such as the Pure Thix® compounds sold by the company Sud-Chemie;

(7) linear (block structure), grafted, or starburst polyurethane polyethers containing in their chain at least one hydrophilic block, which is generally a polyoxyethylene block which may contain between 50 and 1000 oxyethylene units approximately, and at least one hydrophobic block, which may contain aliphatic groups alone, optionally combined with cycloaliphatic and/or aromatic blocks. In various embodiments, the polyurethane polyethers contain at least two C6-C30 hydrocarbon-based hydrophobic chains, separated by a hydrophilic block; the hydrophobic chains may be pendent chains or chains with one or more of the end groups of the hydrophilic block(s).

The polyurethane polyethers may contain a urethane bond between the hydrophilic blocks, but may also contain hydrophilic blocks linked to the lipophilic blocks via other chemical bonds. Representative examples of polyurethane polyethers include Nuvis® FX 1100 (European and US INCI name “Steareth-100/PEG-136/HMDI Copolymer” sold by the company Servo Delden); Rheolate® 205, 208, 204 or 212 (sold by the company Rheox) and also Acrysol RM 184® (sold by the company Rohm & Haas); Elfacos T210® (C12-C14 alkyl chain) and Elfacos T212® (C18 alkyl chain) sold by the company Akzo. The product DW 1206B® from Rohm and Haas containing a C20 alkyl chain and comprising a urethane bond, provided at a solids content of 20% in water, can also be used.

Solutions or dispersions of these polymers, for example in water or in an aqueous/alcoholic medium, may also be useful, such as polymers of Rheolate® 255, Rheolate® 278 and Rheolate® 244, sold by Rheox, and DW 1206F and DW 1206J provided by Rohm and Haas.

The anionic amphiphilic polymers containing a hydrophobic chain that may be used may contain, as the hydrophobic chain, at least one saturated or unsaturated, aromatic or non-aromatic, linear or branched C8-C30 hydrocarbon-based chain.

More particularly, the anionic amphiphilic polymers containing at least one hydrophobic chain which are crosslinked or non-crosslinked, contain at least one hydrophilic unit derived from one or more ethylenically unsaturated monomers bearing a carboxylic acid function, or a sulphonic function which is free or partially or totally neutralized, and at least one hydrophobic unit derived from one or more ethylenically unsaturated monomers bearing a hydrophobic side chain, and optionally at least one crosslinking unit derived from one or more polyunsaturated monomers.

The amphiphilic polymers may also optionally contain at least one sulphonic group, in free or partially- or totally-neutralized form, and at least one hydrophobic portion. Representative examples include acrylamido-2-methyl-2-propanesulphonic (AMPS) acid/n-dodecylacrylamide copolymer neutralized with sodium hydroxide, the copolymer crosslinked with methylenebisacrylamide consisting of 75% by weight of AMPS units neutralized by NH₃ and 25% by weight of Genapol® T-250 acrylate units, the copolymer crosslinked with allyl methacrylate consisting of 90% by weight of AMPS units neutralized with NH₃ and 10% by weight of Genapol® T-250 methacrylate units, or the copolymer crosslinked with allyl methacrylate consisting of 80% by weight of AMPS units neutralized with NH₃ and 20% by weight of Genapol® T-250 methacrylate units.

Other examples include Carbopol® ETD-2020 (acrylic acid/C10-C30 alkyl methacrylate crosslinked copolymer sold by the company Noveon); Carbopol® 1382, Pemulentm™ TR1 and Pemulenm™ TR2 (acrylic acid/C10-C30 alkyl acrylate crosslinked copolymers-sold by the company Noveon), the methacrylic acid/ethyl acrylate/oxyethylenated stearyl methacrylate copolymer (55/35/10); the (meth)acrylic acid/ethyl acrylate/25 EO oxyethylenated behenyl methacrylate copolymer (ACULYN® 28 sold by Rohm & Haas) and the methacrylic acid/ethyl acrylate/steareth-10 allyl ether crosslinked copolymer.

Yet other examples include anionic thickening polymers chosen from crosslinked terpolymers of methacrylic acid, ethyl acrylate, and polyethylene glycol (10 EO) stearyl alcohol ether (Steareth 10), such as the products sold by the company ALLIED COLLOIDS under the names SALCARE® SC 80 and SALCARE® SC 90, which are aqueous emulsions containing 30% of a crosslinked terpolymer of methacrylic acid, of ethyl acrylate and of steareth-10-allyl ether (40/50/10).

Anionic thickening polymers containing at least one fatty chain can also be chosen from: (1) terpolymers formed from maleic anhydride/C30-C38 alpha-olefin/alkyl maleate such as the product (maleic anhydride/C30-C38 alpha-olefin/isopropyl maleate copolymer) sold under the name PERFORMA®1608 by the company NEWPHASE TECHNOLOGIES™; (2) acrylic terpolymers formed from: (a) 20% to 70% by weight of a carboxylic acid with a, β-monoethylenic unsaturation; (b) 20% to 80% by weight of a nonsurfactant monomer with α,β-monoethylenic unsaturation different from (a); and (c) 0.5% to 60% by weight of a nonionic monourethane which is the product of the reaction of a monohydric surfactant with a monoisocyanate with monoethylenic unsaturation; (3) copolymers formed from at least two monomers, wherein at least one of the two monomers is chosen from a carboxylic acid with α,β-monoethylenic unsaturation, an ester of a carboxylic acid with α,β-monoethylenic unsaturation, and an oxyalkylenated fatty alcohol; and (4) copolymers formed from at least three monomers, wherein at least one of the three monomers is chosen from a carboxylic acid with α,β-monoethylenic unsaturation, at least one of the three monomers is chosen from an ester of a carboxylic acid with α,β-monoethylenic unsaturation and at least one of the three monomers is chosen from an oxyalkylenated fatty alcohol.

Additionally, these compounds can also contain, as a monomer, a carboxylic acid ester comprising an α,β-monoethylenic unsaturation and a C1-C4 alcohol. By way of example of this type of compound, there may be mentioned ACULYN® 22 sold by the company ROHM and HAAS, which is an oxyalkylenated stearyl methacrylate/ethyl acrylate/methacrylic acid terpolymer.

Examples of associative polymers are described in WO/2011/076792, and include associative polyurethanes which are cationic or nonionic, associative cellulose derivatives which are cationic or nonionic, associative vinyllactams, associative unsaturated polyacids, associative aminoplast ethers, and associative polymers or copolymers containing at least one monomer comprising ethylenic unsaturation, and containing a sulpho group.

A representative example of an associative polyurethane is methacrylic acid/methyl acrylate/ethoxylated (40 EO) behenyl alcohol dimethyl(meta-isopropenyl)benzyl isocyanate terpolymer as a 25 percent aqueous dispersion, known by the trade name, Viscophobe® DB 1000 and commercially available from Amerchol.

Other examples include polyether polyurethanes capable of being obtained by polycondensation of at least three compounds containing (i) at least one polyethylene glycol containing from 150 to 180 mol of ethylene oxide, (ii) stearyl alcohol or decyl alcohol and (iii) at least one diisocyanate. Such polyether polyurethanes are commercially available from Rohm and Haas under the names ACULYN 46® and ACULYN 44®. ACULYN 46® is a polycondensate of polyethylene glycol comprising 150 or 180 mol of ethylene oxide, of stearyl alcohol and of methylenebis(4-cyclohexyl isocyanate) (SMDI), at 15 percent by weight in a matrix of maltodextrin (4%) and of water. 81% ACULYN 44® is a polycondensate of polyethylene glycol containing 150 or 180 mol of ethylene oxide, of decyl alcohol and of methylenebis(4-cyclohexyl isocyanate) (SMDI), at 35 percent) by weight in a mixture of propylene glycol (39%) and of water (26%).

Representative examples of associative celluloses include quaternized cationic celluloses and quaternized cationic hydroxyethylcelluloses modified by groups containing at least one hydrophobic chain, such as alkyl, arylalkyl or alkylaryl groups containing at least 8 carbon atoms, and mixtures thereof.

The alkyl radicals carried by the above quaternized celluloses or hydroxyethylcelluloses may, in various embodiments, comprise from 8 to 30 carbon atoms. The aryl radicals may, for example, denote the phenyl, benzyl, naphthyl or anthryl groups. Representative examples of quaternized alkylhydroxy-ethylcelluloses containing a C8-C30 hydrophobic chain include the products Quatrisoft LM 200®, Quatrisoft LM-X 529-18-A®, Quatrisoft LM-X 529-18B® (C12 alkyl) and Quatrisoft LM-X 529-8® (C18 alkyl) sold by Amerchol and the products Crodacel QM®, Crodacel QL® (C₁₋₂ alkyl) and Crodacel QS® (C18 alkyl) sold by Croda.

Representative examples of nonionic cellulose derivatives include hydroxyethylcelluloses modified by groups comprising at least one hydrophobic chain, such as alkyl, arylalkyl or alkylaryl groups, or their blends, and in which the alkyl groups are, for example, C8-C22 alkyl groups, such as the product Natrosol Plus Grade 330 CS® (C₁₋₆ alkyls) sold by Aqualon or the product Bermocoll EHM 100® sold by Berol Nobel.

Representative examples of cellulose derivatives modified by alkylphenyl polyalkylene glycol ether groups include the product Amercell Polymer HM-1500® sold by Amerchol.

As regards the associative polyvinyllactams, representative examples include the polymers described in FR 0101106, e.g., poly(vinyllactam) polymers, of vinylpyrrolidone/dimethylaminopropylmethacrylamide/dodecyldimethylmethacrylamidopropylammonium tosylate terpolymers, vinylpyrroidone/dimethylaminopropylmethacrylamide/cocoyldimethylmethacrylamidopropylammonium tosylate terpolymers or vinylpyrrolidone/dimethylaminopropylmethacrylamide/lauryldimethylmethacrylamidopropylammonium tosylate or chloride terpolymers. The vinylpyrrolidone/dimethylaminopropylmethacrylamide/lauryldimethylmethacrylamidopropylammonium chloride terpolymer is provided at 20 percent in water by ISP under the name Styleze® W20.

Associative polyvinyllactam derivatives can also be nonionic copolymers of vinylpyrrolidone and of hydrophobic monomers comprising a hydrophobic chain, for example, the products Antaron V216® or Ganex V2 16® (vinylpyrrolidone/hexadecene copolymer) sold by ISP, or the products Antaron V220® or Ganex V220® (vinylpyrrolidone/eicosene copolymer) sold by ISP.

Examples of associative polymers comprising an aminoplast ether backbone include the products Pure-Thix® L (PEG-180/Octoxynol-40/TMMG Copolymer), Pure-Thix M® (PEG-1 80/Laureth-50/TMMG Copolymer), Pure-Thix® HH (Polyether-1); Pure-Thix TX-1442® (PEG-1 8/dodoxynol-5/PEG-25 tristyrylphenol/tetramethoxymethyl-glyco luril copolymer), which are provided by Sud-Chemie.

Associative polymers may also be chosen from water-soluble thickening polymers. In some embodiments, the rheology-modifying agent may be chosen from thickening polymers comprising at least one fatty chain, such as described in U.S. Pat. No. 7,771,492; thickening polymers chosen from (i) copolymers resulting from the polymerization of at least one monomer (a) chosen from carboxylic acids possessing α,β-ethylinic unsaturation or their esters with at least one monomer (b) possessing ethylinic unsaturation containing a hydrophobic group, (ii) polymers containing at least one monomer possessing a sulpho group, and mixtures thereof, such as described in U.S. Patent Application Publication 2011/0088711A1; rheological agents such as crystalline and semi-crystalline polymers, esters of dextrin and a fatty acid, modified hydrophobic polysaccharides, crystalline olefin copolymers, crystalline polycondensates, lipophilic miners structure-forming agents, lipophilic polyamide polymers, lipophilic polyureas and polyurethanes, silicone polymers, organic gelling agents, block copolymers, silicone elastomers, cholesteric liquid crystal agents, waxes, and mixtures thereof, such as described in U.S. Patent Application Publication 2011/0200543 A1; and non-associative thickening polymers, such as described in U.S. Pat. No. 7,250,064.

The rheology-modifying agent is typically present in an amount ranging from about 0.01% to about 10% by weight, in some embodiments from about 0.1% to about 5% by weight, based on the total weight of the composition.

### Fatty Phase Ingredients

In addition to the aqueous solvent or the aqueous/organic solvent combination, at least one cosmetically or dermatologically acceptable and, in general, physiologically acceptable oil may be present. As used herein, the term “oil” means any fatty substance that is in liquid form at room temperature and atmospheric pressure. Oils that may be suitable for use in the present invention include both volatile and nonvolatile oils.

The volatile or nonvolatile oils are typically selected from hydrocarbon-based oils, silicone oils, and fluoro oils. The term “hydrocarbon-based oil” refers to oil mainly containing hydrogen and carbon atoms and possibly oxygen, nitrogen, sulfur and/or phosphorus atoms.

Representative categories of non-volatile hydrocarbon-based oils include fatty acids, linear or branched hydrocarbons of plant, mineral, or plant origin, and synthetic oils such as esters and ethers, fatty alcohols and fatty amides.

Examples of fatty acids include caprylic acid, capric acid, lauric acid, myristic acid, palmitic acid, stearic acid, oleic acid, ricinoleic acid, linoleic acid, linolenic acid, arachidic acid, gadoleic acid, behenic acid, erucic acid, brassidic acid, cetoleic acid, lignoceric acid and nervonic acid.

Examples of linear or branched hydrocarbons of mineral origin include mineral oils (e.g., paraffin), petroleum jelly, polydecenes, hydrogenated polyisobutene such as Parleam, perhydrosqualene and squalane.

Examples of hydrocarbon-based plant oils include triglycerides consisting of fatty acid esters of glycerol, the fatty acids of which may have chain lengths ranging from C4 to C24, these chains possibly being linear or branched, and saturated or unsaturated, e.g., heptanoic or octanoic triglycerides, groundnut oil, babassu oil, coconut oil, grapeseed oil, cottonseed oil, corn oil, corn germ oil, mustard seed oil, palm oil, rapeseed oil, sesame seed oil, soybean oil, sunflower oil, wheatgerm oil, canola oil, apricot oil, mango oil, castor oil, shea oil, avocado oil, olive oil, sweet almond oil, peach kernel oil, walnut oil, hazelnut oil, macadamia oil, jojoba oil, alfalfa oil, poppy seed oil, pumpkin oil, marrow oil, blackcurrant seed oil, evening primrose oil, millet oil, barley oil, quinoa oil, rye oil, safflower oil, candlenut oil, passionflower oil, musk rose oil or shea butter oil and alternatively caprylic/capric acid triglycerides.

Representative examples of synthetic esters and ethers, in particular of fatty acids, such as oils of formulae R1COOR2 and R1-OR2 in which R1 represents the residue of a fatty acid or of a fatty alcohol comprising from 8 to 29 carbon atoms and R2 represents a branched or unbranched hydrocarbon chain comprising from 3 to 30 carbon atoms, such as, for example, purcellin oil, octyl palmitate, isopropyl lanolate, 2-octyldodecyl stearate, 2-octyldodecyl erucate or isostearyl isostearate; hydroxylated esters, such as isostearyl lactate, octyl hydroxystearate, octyldodecyl hydroxystearate, diisostearyl malate, triisocetyl citrate or heptanoates, octanoates or decanoates of fatty alcohols; polyol esters, such as propylene glycol dioctanoate, neopentyl glycol diheptanoate and diethylene glycol diisononanoate; and pentaerythritol esters, such as pentaerythrityl tetraisostearate; or lipophilic derivatives of amino acids, such as isopropyl lauroyl sarcosinate (INCI name). Yet other examples include C12-C15 alkyl benzoates such as those sold under the tradenames “Finsolv TN” and “Witconol TN” by the company Witco, and 2-ethylphenyl benzoate, for instance the product sold under the name X-TEND 226® by the company ISP, triglycerides such as dicaprylyl carbonate (e.g., Cetiol CC, sold by Cognis), and oxyethylenated or oxypropylenated fatty esters and ethers.

Fatty alcohols which may be useful in the present invention tend to be liquid at room temperature and have a branched and/or unsaturated carbon-based chain containing from 12 to 26 carbon atoms. Representative examples thus include 2-octyldodecanol, isostearyl alcohol, oleyl alcohol, 2-hexyldecanol, 2-butyloctanol and 2-undecylpentadecanol.

Representative examples of fatty amides include isopropyl lauroyl sarcosinate such as the product sold under the tradename “Eldew SL-205” by the company Ajinomoto).

Representative examples of volatile hydrocarbon-based oils include oils containing from 8 to 16 carbon atoms, and especially branched C8-C16 alkanes (also known as isoparaffins), for instance isododecane (also known as 2,2,4,4,6-pentamethylheptane), isodecane and isohexadecane.

Examples of nonvolatile silicone oils that may be useful in the present invention include nonvolatile polydimethylsiloxanes (PDMS), polydimethylsiloxanes comprising alkyl or alkoxy groups that are pendent and/or at the end of a silicone chain, these groups each containing from 2 to 24 carbon atoms, phenyl silicones, for instance phenyl trimethicones, phenyl dimethicones, phenyl trimethylsiloxy diphenylsiloxanes, diphenyl dimethicones, diphenyl methyldiphenyl trisiloxanes and 2-phenylethyl trimethylsiloxysilicates, and dimethicones or phenyltrimethicones with a viscosity of less than or equal to 100 cSt.

Representative examples of volatile silicone oils that may be useful in the present invention include volatile linear or cyclic silicone oils, especially those with a viscosity ÿ centistokes (8×10-6 m 2/s) and especially containing from 2 to 10 silicon atoms and in particular from 2 to 7 silicon atoms, these silicones optionally comprising alkyl or alkoxy groups containing from 1 to 10 carbon atoms. Specific examples include dimethicones with a viscosity of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

Representative examples of volatile fluoro oils that may be suitable for use in the present invention include nonafluoromethoxybutane and perfluoro-methylcyclopentane.

The amount of oil that may present in the compositions generally ranges from about 5% to about 99% and in some embodiments, from about 10% to about 80% by weight, based on the total weight of the composition.

The fatty phase may contain any other cosmetically acceptable fat-soluble or fat/oil-dispersible additive such as waxes and other polymeric structuring agents, and pasty compounds or substances, which as used herein, refer to fatty compounds with a reversible solid/liquid change of state and containing, at a temperature of 25° C., a liquid fraction and a solid fraction. Examples of pasty compounds, such as polyol esters, are described in U.S. Patent Application Publication 2010/0015074 A1. Suitable waxes are those generally used in cosmetics and dermatology. Representative examples of waxes include those of natural animal, plant or mineral origin, for instance beeswax, carnauba wax, candelilla wax, ouricury wax, Japan wax, cork fiber wax, sugar cane wax, paraffin wax, lignite wax, microcrystalline waxes, lanolin wax, montan wax, ozokerites and hydrogenated oils such as hydrogenated jojoba oil as well as waxes of synthetic origin, for instance polyethylene waxes derived from the polymerization of ethylene, waxes obtained by Fischer-Tropsch synthesis, fatty acid esters and glycerides that are solid at 40° C., for example, at above 55° C., silicone waxes such as alkyl- and alkoxy-poly(di)methylsiloxanes and/or poly(di)methyl-siloxane esters that are solid at 40° C., for example, at above 55° C. Waxes approved for food use include ozokerite, rice wax and the waxes referenced in the Codex alimentary.

The amount of fatty phase (including both liquids and solids), exclusive of emulsifier and hydrophobic gelling agent, that may present in the compositions generally ranges from about 5% to about 80% and in some embodiments, from about 10% to about 50% by weight, based on the total weight of the composition.

### Emulsifier/Surfactant

Representative examples of emulsifiers and surfactants that may be useful in the practice of the present invention include non-ionic amphiphilic lipids and anionic amphiphilic lipids.

Nonionic Amphiphilic Lipids: Representative examples of nonionic amphiphilic lipids include 1) silicone surfactants; 2) amphiphilic lipids that are fluid at a temperature of less than or equal to 45° C., chosen from the esters of at least one polyol chosen from the group formed by polyethylene glycol containing from 1 to 60 ethylene oxide units, sorbitan, glycerol containing from 2 to 30 ethylene oxide units, polyglycerols containing from 2 to 15 glycerol units, and of at least one fatty acid containing at least one saturated or unsaturated, linear or branched C8-C22 alkyl chain; 3) mixed esters of fatty acid or of fatty alcohol, of carboxylic acid and of glycerol; 4) fatty acid esters of sugars and fatty alcohol ethers of sugars; 5) surfactants that are solid at a temperature of less than or equal to 45° C., chosen from fatty esters of glycerol, fatty esters of sorbitan and oxyethylenated fatty esters of sorbitan, ethoxylated fatty ethers and ethoxylated fatty esters; and 6) block copolymers of ethylene oxide (A) and of propylene oxide (B).

Representative examples of silicone surfactants include silicone compounds comprising at least one oxyethylene chain —OCH₂CH₂— and/or oxypropylene chain —OCH₂CH₂CH₂—, as described in U.S. Pat. Nos. 5,364,633 and 5,411,744.

Representative silicone surfactants also include compounds of formula (I):

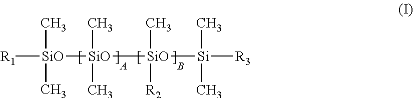

in which:
R₁, R₂ and R₃, independently of each other, represent a C1-C6 alkyl radical or a radical —(CH₂)ₓ— (OCH₂CH₂)_{y}—(OCH₂CH₂CH₂)_{z}—OR₄, at least one radical R₁, R₂ or R₃ not being an alkyl radical; R₄ being a hydrogen, an alkyl radical or an acyl radical; A is an integer ranging from 0 to 200; B is an integer ranging from 0 to 50; with the proviso that A and B are not simultaneously equal to zero; x is an integer ranging from 1 to 6; y is an integer ranging from 1 to 30; and z is an integer ranging from 0 to 5. In some embodiments, in the compound of formula (I), the alkyl radical is a methyl radical, x is an integer ranging from 2 to 6 and y is an integer ranging from 4 to 30.

Specific examples of silicone surfactants of formula (I) include the compounds of formula (II):

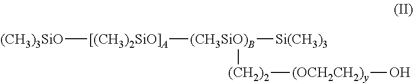

in which A is an integer ranging from 20 to 105, B is an integer ranging from 2 to 10 and y is an integer ranging from 10 to 20.

Other examples of silicone surfactants of formula (I) include the compounds of formula (III):

H—(OCH₂CH₂)y—(CH₂)₃—[(CH₃)₂SiO]A′-(CH₂)₃—(OCH₂CH₂)y-OH (III)

in which A′ and y are integers ranging from 10 to 20.

Compounds of the invention which may be used are commercially available from Dow Corning under the tradenames DC 5329, DC 7439-146, DC2-5695 and Q4-3667. The compounds DC 5329, DC 7439-146 and DC2-5695 are compounds of formula (II) in which, respectively, A is 22, B is 2 and y is 12; A is 103, B is 10 and y is 12; A is 27, B is 3 and y is 12. The compound Q4-3667 is a compound of formula (III) in which A is 15 and y is 13. Other compounds may include PEG-12/-14 dimethicones or silsoft 880 etc. from GE/Momentive Materials. These can also provide placistization of styling films.

Representative examples of amphiphilic lipids that are fluid at a temperature of less than or equal to 45° C. include the following: the isostearate of polyethylene glycol of molecular weight 400, sold under the name PEG 400 by the company Unichema; diglyceryl isostearate, sold by the company Solvay; glyceryl laurate comprising 2 glycerol units, sold by the company Solvay; sorbitan oleate, sold under the name Span 80 by the company ICI; sorbitan isostearate, sold under the name Nikkol SI 10R by the company Nikko; and α-butylglucoside cocoate or α-butylglucoside caprate, sold by the company Ulice.

Representative examples of mixed esters of fatty acid or of fatty alcohol, of carboxylic acid and of glycerol include mixed esters of fatty acid or of fatty alcohol with an alkyl chain containing from 8 to 22 carbon atoms, and of α-hydroxy acid and/or of succinic acid, with glycerol. The α-hydroxy acid may be, for example, citric acid, lactic acid, glycolic acid or malic acid, and mixtures thereof.

The alkyl chain of the fatty acids or alcohols from which are derived the mixed esters may be linear or branched, and saturated or unsaturated. They may be stearate, isostearate, linoleate, oleate, behenate, arachidonate, palmitate, myristate, laurate, caprate, isostearyl, stearyl, linoleyl, oleyl, behenyl, myristyl, lauryl or capryl chains, and mixtures thereof.

Representative examples of mixed esters include the following: the mixed ester of glycerol and of the mixture of citric acid, lactic acid, linoleic acid and oleic acid (CTFA name: Glyceryl citrate/lactate/linoleate/oleate) sold by the company Hills under the name Imwitor® 375; the mixed ester of succinic acid and of isostearyl alcohol with glycerol (CTFA name: Isostearyl diglyceryl succinate) sold by the company Hills under the name Imwitor® 780K; the mixed ester of citric acid and of stearic acid with glycerol (CTFA name: Glyceryl stearate citrate) sold by the company Hills under the name Imwitor® 370; the mixed ester of lactic acid and of stearic acid with glycerol (CTFA name: Glyceryl stearate lactate) sold by the company Danisco under the name Lactodan B30 or Rylo LA30.

Representative examples of fatty acid esters of sugars, which are typically solid at a temperature of less than or equal to 45° C., include esters or mixtures of esters of C8-C22 fatty acid and of sucrose, of maltose, of glucose or of fructose, and esters or mixtures of esters of C14-C22 fatty acid and of methylglucose.

The C8-C22 or C14-C22 fatty acids forming the fatty unit of the esters contain a saturated or unsaturated linear alkyl chain containing, respectively, from 8 to 22 or from 14 to 22 carbon atoms. The fatty unit of the esters may be chosen from stearates, behenates, arachidonates, palmitates, myristates, laurates and caprates, and mixtures thereof.

Specific examples of esters or mixtures of esters of fatty acid and of sucrose, of maltose, of glucose or of fructose include sucrose mono-stearate, sucrose distearate and sucrose tristearate and mixtures thereof, such as the products sold by the company Croda under the name Crodesta F50, F70, F110 and F160 having, respectively, an HLB (hydrophilic lipophilic balance) of 5, 7, 11 and 16; and examples of esters or mixtures of esters of fatty acid and of methylglucose which may be mentioned are methylglucose polyglyceryl-3 distearate, sold by the company Goldschmidt under the name Tego® Care 450. Mention may also be made of glucose or maltose monoesters such as methyl o-hexadecanoyl-6-D-glucoside and o-hexadecanoyl-6-D-maltoside.

Representative examples of fatty alcohol ethers of sugars, which are typically solid at a temperature of less than or equal to 45° C., include ethers or mixtures of ethers of C8-C22 fatty alcohol and of glucose, of maltose, of sucrose or of fructose, and ethers or mixtures of ethers of a C14-C22 fatty alcohol and of methylglucose, e.g., alkylpolyglucosides.

The C8-C22 or C14-C22 fatty alcohols forming the fatty unit of the ethers contain a saturated or unsaturated, linear alkyl chain containing, respectively, from 8 to 22 or from 14 to 22 carbon atoms. The fatty unit of the ethers may be chosen from decyl, cetyl, behenyl, arachidyl, stearyl, palmityl, myristyl, lauryl, capryl and hexa-decanoyl units, and mixtures thereof, such as cetearyl.

Specific examples of fatty alcohol ethers of sugars include alkylpolyglucosides such as decylglucoside and laurylglucoside, which is sold, for example, by the company Henkel under the respective names Plantaren® 2000 and Plantaren® 1200, cetostearyl glucoside optionally as a mixture with cetostearyl alcohol, sold for example, under the name Montanov 68 by the company SEPPIC, under the name Tego® Care CG90 by the company Goldschmidt and under the name Emulgade® KE3302 by the company Henkel, as well as arachidyl glucoside, for example in the form of a mixture of arachidyl alcohol and behenyl alcohol and arachidyl glucoside, sold under the name Montanov™ 202 by the company SEPPIC. In some embodiments, the surfactant is sucrose monostearate, sucrose distearate or sucrose tristearate and mixtures thereof, methylglucose poly-glyceryl-3 distearate and alkylpolyglucosides.

Representative examples of fatty esters of glycerol, which are typically solid at a temperature of less than or equal to 45° C., include esters formed from at least one acid comprising a saturated linear alkyl chain containing from 16 to 22 carbon atoms and from 1 to 10 glycerol units, such as stearates, behenates, arachidates and palmitates, and mixtures thereof. Other examples include decaglyceryl monostearate, distearate, tristearate and pentastearate (CTFA names: Poly-glyceryl-10 stearate, Polyglyceryl-10 distearate, Poly-glyceryl-10 tristearate, Polyglyceryl-10 penta-stearate), such as the products sold under the respective names Nikkol Decaglyn 1-S, 2-S, 3-S and 5-S by the company Nikko, and diglyceryl monostearate (CTFA name: Polyglyceryl-2 stearate), such as the product sold by the company Nikko under the name Nikkol DGMS.

Representative examples of fatty esters of sorbitan, which are typically solid at a temperature of less than or equal to 45° C., include C16-C22 fatty acid esters of sorbitan and oxyethylenated C16-C22 fatty acid esters of sorbitan. They are formed from at least one fatty acid containing at least one saturated linear alkyl chain containing, respectively, from 16 to 22 carbon atoms, e.g., stearates, behenates, arachidates, palmitates, and mixtures thereof, and from sorbitol or from ethoxylated sorbitol. The oxy-ethylenated esters generally contain from 1 to 100 ethylene glycol units and preferably from 2 to 40 ethylene oxide (EO) units. Specific examples include sorbitan monostearate (CTFA name: sorbitan stearate), sold by the company ICI under the name Span™ 60, sorbitan monopalmitate (CTFA name: sorbitan palmitate), sold by the company ICI under the name Span™ 40, and sorbitan tristearate 20 EO (CTFA name: poly-sorbate 65), sold by the company ICI under the name Tween 65.

Representative examples of ethoxylated fatty ethers, which are typically solid at a temperature of less than or equal to 45° C., include ethers formed from 1 to 100 ethylene oxide units and from at least one fatty alcohol chain containing from 16 to 22 carbon atoms. The fatty chain of the ethers may be chosen from behenyl, arachidyl, stearyl and cetyl units, and mixtures thereof, such as cetearyl. Specific examples of ethoxylated fatty ethers include behenyl alcohol ethers containing 5, 10, 20 and 30 ethylene oxide units (CTFA names: beheneth-5, beheneth-10, beheneth-20, beheneth-30), such as the products sold under the names Nikkol BB5, BB10, BB20 and BB30 by the company Nikko, and stearyl alcohol ether comprising 2 ethylene oxide units (CTFA name: steareth-2), such as the product sold under the name Brij® 72 by the company ICI.

Representative examples of ethoxylated fatty esters, which are typically solid at a temperature of less than or equal to 45° C., include esters formed from 1 to 100 ethylene oxide units and from at least one fatty acid chain containing from 16 to 22 carbon atoms. The fatty chain in the esters may be chosen from stearate, behenate, arachidate and palmitate units, and mixtures thereof. Specific examples of ethoxylated fatty esters include the ester of stearic acid containing 40 ethylene oxide units, such as the product sold under the name Myrjm™ 52 (CTFA name: PEG-40 stearate) by the company ICI, as well as the ester of behenic acid containing 8 ethylene oxide units (CTFA name: PEG-8 behenate), such as the product sold under the name Compritol HD5 ATO by the company Gattefosse.

Representative examples of block copolymers of ethylene oxide (A) and of propylene oxide (B), include the block copolymers of formula (IV):

HO(C₂H₄O)ₓ(C₃H₆O)_{y}(C₂H₄O)_{y}H (IV)

in which x, y and z are integers such that x+z ranges from 2 to 100 and y ranges from 14 to 60, and mixtures thereof, and more particularly from the block copolymers of formula (IV) having an HLB value ranging from 2 to 16. These block copolymers include poloxamers such as Poloxamer 231, e.g., the product sold by ICI under the name Pluronic L81 (wherein x=z=6, y=39 (HLB 2)); Poloxamer 282, such as the product sold by ICI under the name Pluronic L92 (wherein x=z=10, y=47 (HLB 6)); and Poloxamer 124, such as the product sold by ICI under the name Pluronic® L44 (wherein x=z=11, y=21 (HLB 16).

In some embodiments, the nonionic amphiphilic lipid is polyethylene glycol isostearate (8 mol of ethylene oxide), diglyceryl isostearate, polyglyceryl monolaurate and monostearate comprising 10 glycerol units, sorbitan oleate, and or sorbitan isostearate.

### Anionic Amphiphilic Lipids:

Representative anionic amphiphilic lipids include alkyl ether citrates, alkoxylated alkenyl succinates, alkoxylated glucose alkenyl succinates, and alkoxylated methylglucose alkenyl succinates.

Representative examples of alkyl ether citrates include monoesters, diesters or triesters formed from citric acid and from at least one oxyethylenated fatty alcohol containing a linear or branched, saturated or unsaturated alkyl chain containing from 8 to 22 carbon atoms, and containing from 3 to 9 ethoxylated groups, and mixtures thereof. These citrates may be chosen, for example, from the mono-, di- and triesters of citric acid and of ethoxylated lauryl alcohol, containing from 3 to 9 ethoxylated groups, which are sold by the company Witco under the name Witconol™ EC, in particular Witconol™ EC 2129 which is predominantly a dilaureth-9 citrate, and Witconol™ EC 3129 which is predominantly a trilaureth-9 citrate.

The alkyl ether citrates may be formulated for use at a pH of about 7, using a neutralizing agent such as an inorganic base e.g., sodium hydroxide, potassium hydroxide and ammonia, or an organic base, e.g., mono-ethanolamine, diethanolamine, triethanolamine, 1,3-aminomethylpropanediol, N-methylglucamine, and a basic amino acid (e.g, arginine and lysine), and mixtures thereof.

Representative examples of alkenyl succinates incude ethoxylated and/or propoxylated derivatives such as the compounds of formula (V) and (VI):

HOOC—(HR)C—CH2-COO-E (V)

HOOC—(HR)C—CH2-COO-E-O—CO—CH2-C(HR′)—COOH (VI)

in which the radicals R and R′ are chosen from linear or branched alkenyl radicals containing from 6 to 22 carbon atoms, and E is chosen from oxyethylene chains of formula (C₂H₄O)ₙ in which n ranges from 2 to 100, oxy-propylene chains of formula (C₃H₆O)_{n′} in which n′ ranges from 2 to 100, random or block copolymers containing oxyethylene chains of formula (C₂H₄O)ₙ and oxypropylene chains of formula (C₃H₆O)ₙ, such that the sum of n and n′ ranges from 2 to 100, the oxyethylenated and/or oxypropylenated glucose groups containing on average from 4 to 100 oxyethylene and/or oxypropylene units distributed on all the hydroxyl functions, the oxyethylenated and/or oxypropylenated methylglucose groups containing on average from 4 to 100 oxyethylene and/or oxypropylene units distributed on all the hydroxyl functions.

In formulae (V) and (VI), n and n′ are average values and are thus not necessarily integers. A value of n ranging from 5 to 60 and in some embodiments from 10 to 30 is advantageously chosen.

The radical R and/or R′ may be chosen from linear alkenyl radicals containing from 8 to 22 and in some embodiments from 14 to 22 carbon atoms. It may be, for example, the hexadecenyl radical containing 16 carbon atoms or the octadecenyl radical containing 18 carbon atoms.

The compounds of formulae (V) and (VI) described above, in which E is chosen from oxyethylene chains, oxypropylene chains and copolymers containing oxyethylene chains and oxypropylene chains, may be prepared in accordance with the description given in WO-A-94/00508, EP-A-107199 and GB-A-2 131 820.

The acid function —COOH in the surfactants of formulae (V) and (VI) may be neutralized with a neutralizing agent such as those described above in connection with the surfactants of formula IV.

Representative examples of these surfactants include hexadecenyl succinate 18 EO (compound of formula V wherein R=hexadecenyl, E=(C₂H₄O)ₙ, n=18), hexa-decenyl succinate 45 EO (compound of formula V wherein R=hexadecenyl, E=(C₂H₄O)ₙ, n=45), dihexadecenyl succinate 18 EO (compound of formula VI wherein R═R′=hexadecenyl, E=(C₂H₄O)ₙ, n=18), dihexadecenyl glucose succinate 10 EO (compound of formula VI wherein R═R′=hexadecenyl, E=oxyethylenated glucose containing 10 oxyethylene groups), dihexadecenyl glucose succinate 20 EO (compound of formula VI wherein R═R′=hexadecenyl, E=oxyethylenated glucose containing 20 oxyethylene groups), dioctadecenyl methylglucose succinate 20 EO (compound of formula VI wherein R═R′=octadecenyl, E=oxyethylenated methylglucose containing 20 oxy-ethylene groups), and mixtures thereof.

Depending on its more hydrophilic or more lipophilic nature, the nonionic or anionic amphiphilic lipid may be introduced into the aqueous phase or (if present) into the oily phase of the cosmetic composition.

Other Emulsifiers/Surfactants: Cationic and amphoteric surfactants or emulsifiers may also be useful. Examples of cationic emulsifiers include salts of primary, secondary or tertiary fatty amines, optionally polyoxyalkylenated, and quaternary ammonium salts. Examples of amphoteric emulsifiers include secondary or tertiary aliphatic amine derivatives in which the aliphatic group is a linear or branched chain containing from 8 to 22 carbon atoms and containing at least one anionic group such as, for example, a carboxylate, sulphonate, sulphate, phosphate or phosphonate group. Examples of amine derivatives include amphocarboxyglycinate, amphocarboxypropionate, disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, cocoamphodipropionic acid. They may be available commercially from Rhodia under the tradename MIRANOL® (e.g., MIRANOL® C2M). Other examples of amphoteric emulsifiers include (C₈₋₂₀)alkyl betaines, suiphobetaines, (C8-20 alkyl)amido (C6-8 alkyl)betaines and (C8-20 alkyl)amido(C6-8 alkyl) sulphobetaines.

Emulsifiers/surfactants may be present even in embodiments that are not emulsions. The amount of emulsifier generally ranges from about 0.1% to about 25% and in some embodiments from about 1% to about 20% by weight, based on the total weight of the composition.

### Photoprotectants

These ingredients which are also referred to as U.V. filters, can be organic or inorganic (or physical) agents. Representative examples of commercially available organic and inorganic photoprotective agents that may be useful in the present invention are disclosed, for example, in commonly owned U.S. Patent Application Publication 2010/0190740 A1. Some specific examples of organic photoprotective agents include octocrylene, homosalate, butylmethoxydibenzoylmethane (avobenzone), phenylbenzimidazole sulphonic acid (Eusolex® 232, available from EMD Chemicals, Inc.), ethylhexyl methoxycinnamate, and terephthalylidene dicamphor sulfonic acid (Mexoryl® SX, available from L'Oreal). Inorganic photoprotectants are typically pigments formed of metal oxides which may or may not be coated (and which typically have a mean particle size between about 5×10⁻³ m and 100×10⁻³ m. Specific examples include pigments formed of titanium oxide, iron oxide, zinc oxide, zirconium oxide, and cerium oxide.

Photoprotectants are generally present in an amount ranging from about 1% to about 30% by weight, and in some embodiments from about 1% to about 25% by weight, based on the total weight of the composition.

Representative examples of cosmetically active agents include antidandruff agents (for example, zinc pyrithione, octopirox, selenium sulphide, ellagic acid and derivatives), agents for combating hair loss or agents for promoting hair regrowth, anti-inflammatory agents, vitamins, keratolytic and desquamating agents, α-hydroxy acids, depigmenting agents, salicylic acid, retinoids, hydrocortisone, natural extracts, steroids, anti-bacterial agents, enzymes, flavanoids, soothing agents, mattifying agents, moisturizing agents (e.g., hydrolyzed silk or wheat protein, panthenol), trace elements and essential fatty acids.

Cosmetic active agents and other cosmetic additives may present in the compositions in amounts generally ranging from about 0.01% to about 10% and in some embodiments from about 0.05% to about 8% by weight, based on the total weight of the composition.

The compositions may be applied to wet or dry hair, before or after shaping. They may be used in a non-rinse fashion in order to impart or maintain the shape of the hair. In some other embodiments, the composition may be rinsed from the hair. In some embodiments, following application of the composition, the hair is dried (e.g., air or blow dried), optionally in conjunction with use of shaping tools such as a hot iron.

Embodiments of the present invention will now be described in terms of the following non-limiting working examples. Unless indicated to the contrary, all parts are by weight.

The following inventive compositions were prepared.

TABLE 1

| | % weight | | | | | |
|---|---|---|---|---|---|---|
| Ingredients | Ex. 1 | Ex. 2 | Ex. 3 | Ex. 4 | Ex. 5 | Ex. 6 |
| Deionized | 39.148 | 40.8 | 41.848 | 40.80 | 95.33 | 39.23 |
| Water | | | | | | |
| Ethanol | 48.16 | 51.22 | 51.50 | 51.50 | 0 | 48.16 |
| AMP | 1.14 | 0.78 | 0.50 | 0.50 | 1.22 | 1.14 |
| Gantrez ® | 11.4 | 6 | 6 | 6 | — | 11.4 |
| ES-425L | | | | | | |
| Gantrez ® | — | — | — | — | 3 | — |
| S-97 | | | | | | |
| Polylysine | 0.152 | 1.2 | 0.152 | 1.2 | 0.45 | 0 |
| (25%) | | | | | | |
| Chitosan | | | | | | 0.075 |
| TOTAL | 100 | 100 | 100 | 100 | 100 | 100 |

About 0.5 g of the of each of the formulations described above were applied on 1 g of hair fiber which was then folded on the curly plastic hair loops and dried at 50° C. for 1 hr. The treated samples were then left to air dry overnight. The next day they were inserted in a humidity chamber for the High Humidity Curl Retention Test (HHCR) at a specified Relative Humidity and Temperature. The percent loop retention was plotted using the equation:

$$\% Curl Retention = \frac{( Lo - Lt )}{( Lo - Li )} * 100$$

wherein Lo=Original hair length (fully extended hair length); Li=Initial hair length (length of hair before exposure); and Lt=Length of hair after exposure. The results are shown in the following table.

TABLE II

| | | | | | Ratio | | | | | |
|---|---|---|---|---|---|---|---|---|---|---|
| | | | | | (wt % | | | | | |
| | | | | | poly- | | | | | |
| | | | | | amino | | | | | |
| | | Solids | | | acid | pH | | | | Curl |
| | | (wt | | Poly- | per | (g | VOC | | | Retention |
| | Polymer | %) | Amine | amine | polymer) | AMP) | (%) | Clarity | Results | (8 hrs) |
| Control | Gantrez ® | 6 | AMP | | 0.0019 | 7 | 55 | Clear | Ref | 55 |
| | ES- | | | | | | | | | |
| | 425L | | | | | | | | | |
| 1 | Gantrez ® | 6 | | ε-polylysine | 0.0066 | 7 | 55 | Clear | Strong | 83 |
| | ES- | | | | | | | | complex | |
| | 425L | | | | | | | | | |
| 2 | Gantrez ® | 3 | | ε-polylysine | 0.10 | 7 | 55 | Clear | Strong | 91 |
| | ES- | | | | | | | | complex | |
| | 425L | | | | | | | | | |
| 3 | Gantrez ® | 3 | | ε-polylysine | 0.013 | 7 | 55 | Clear | Strong | 88 |
| | ES- | | | | | | | | complex | |
| | 425L | | | | | | | | | |
| 4 | Gantrez ® | 3 | | ε-polylysine | 0.10 | 7 | 55 | Clear | Strong | 95 |
| | ES- | | | | | | | | complex | |
| | 425L | | | | | | | | | |
| 5 | Gantrez ® | 3 | | ε-polylysine | 0.0375 | 5 | 0 | Clear | Strong | 50 |
| | S- | | | | | | | | complex | |
| | 97 | | | | | | | | | |
| Control | Gantrez ® | 3 | AMP | | 0.0038 | 5 | 0 | Clear | Ref | 25 |
| | S- | | | | | | | | | |
| | 97 | | | | | | | | | |
| 6 | Gantrez ® | 6 | | Kytamer ® | 0.0125 | 7.5 | 55 | Clear/Particles | Complex | 70 |
| | ES- | | | PC | | | | | | |
| | 425L | | | | | | | | | |

The results shown in table II along with the curl retention data, are summarized as follows: (1) Gantrez® ES-425L with E-polylysine (PL) showed a 51% improved humidity resistance versus control Gantrez® ES-425L at 8 hrs (and also 22 hrs) and 80% RH/25C; (2) decreasing the amount of resin to 3% increased HHCR by 60-65-73% for samples 3, 2 and 4 respectively versus the control with 6% resin; (3) increasing the amount of E-polylysine increased the curl retention at the same resin level (samples 3, 2 and 4 respectively); (4) Kytamer® PC increased HHCR by 27% versus control; and (5) S-97 resin and PL increased the HHCR by 50% (no flat iron was employed in these samples).

All publications cited in the specification, both patent publications and non-patent publications, are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A cosmetic composition, comprising: a maleic acid copolymer; a polyamine selected from polyamino acids and aminated polysaccharides; a neutralizer; and water, wherein the polyamine is present in an amount less than 0.1% by weight based on the total weight of the composition.

2. The cosmetic composition of claim 1, wherein the maleic acid copolymer comprises repeating units of maleic acid or maleic anhydride or a derivative of maleic acid or maleic anhydride, and a monomer co-polymerizable therewith, wherein the monomer is selected from the group consisting of vinyl esters, vinyl ethers, vinyl halides, phenylvinyl derivatives, alpha-olefins, cycloolefins, and vinyl triazoles.

3. The composition of claim 1, wherein the maleic acid copolymer is selected from the group consisting of methyl vinyl ether-maleic acid copolymer, methyl vinyl ether-maleic anhydride copolymer (PVM/MA copolymer), ethyl ester of PVM/MA copolymer, butyl ester of PVM/MA copolymer isobutyl vinyl ether-maleic anhydride copolymer, ethylene-maleic anhydride copolymer, isobutylene-maleic anhydride copolymer, styrene-maleic anhydride copolymer, vinyltriazole-maleic anhydride copolymer and vinylnorbornene-maleic anhydride copolymer.

4. The composition of claim 1, wherein the maleic acid copolymer comprises a maleic acid monomer that is partially or fully esterified.

5. The composition of claim 4, wherein the maleic acid copolymer an ethylvinylether/maleic acid copolymer or a butylvinylether/maleic acid copolymer.

6. The composition of claim 1, wherein the maleic acid copolymer comprises a maleic acid monomer that is in the form of a salt or a mixed salt of an alkali metal or an alkaline earther metal.

7. The composition of claim 6, wherein the maleic acid copolymer is calcium/sodium PVM/MA copolymer.

8. The composition of claim 1, wherein the maleic acid copolymer comprises a malemic acid or a maleimide repeating unit that is N-derivatized with a hydrophobic amine, a hydrophilic amine or a polyether amine.

9. The composition of claim 8, wherein the maleic acid copolymer is polyimide-1 or isobutylene/ethylmaleimide/hydroxyethylmaleimide copolymer.

10. The composition of claim 1, wherein the maleic acid copolymer is present in an amount of from greater than 0 to about 12% on a dry weight basis, based on the total weight of the composition.

11. The composition of claim 1, wherein the polyamino acid comprises a positively charged amino acid.

12. The composition of claim 11, wherein the polyamino acid is selected from the group consisting of polylysine, poly-ornithine, polyhomoarginine, polyarginine, poly-glutamic acid and polyhistidine.

13. The composition of claim 11, wherein the polyamino acid is E-Poly-L-lysine.

14. The composition of claim 1, wherein the aminated polysaccharide comprises chitosan or a cosmetically acceptable derivative thereof.

15. The composition of claim 1, wherein the polyamine is present in an amount of from about 0.02% to about 0.04%, on a dry weight basis, based on the total weight of the composition.

16. The composition of claim 1, wherein the polyamine is present in an amount of from about 0.006 g to about 0.04 g, on a dry weight basis, per gram of the maleic acid copolymer.

17. The composition of claim 1, further comprising at least one additional cosmetically acceptable ingredient selected from the group consisting of volatile organic compounds, propellants, rheology modifiers, emulsifiers, surfactants, fatty phase ingredients, preservatives and fragrances, and combinations of two or more thereof.

18. The composition of claim 17, which further comprises a propellant.

19. The composition of claim 18, which further comprises a volatile organic solvent.

20. A method of imparting shape to hair or maintaining shape of hair, comprising applying to hair a cosmetic composition, comprising: a maleic acid copolymer; a polyamine selected from polyamino acids and aminated polysaccharides; a neutralizer; and water, wherein the polyamine is present in an amount less than 0.1% by weight based on the total weight of the composition.
